(19) United States
(12) Patent Application Publication
(10) Pub. No.: US 2016/0262754 A1
(43) Pub. Date: Sep. 15, 2016

(54) FLEXIBLE TACK GUIDE

(71) Applicant: Nir Altman, Kibbutz Kfar Etzion (IL)

(72) Inventor: Nir Altman, Kibbutz Kfar Etzion (IL)

(21) Appl. No.: 14/645,597

(22) Filed: Mar. 12, 2015

(51) Int. Cl. A61B 17/10

(52) U.S. Cl. CPC A61B 17/10

(57) ABSTRACT

A flexible tack guide includes a tube with a wall thickness and having a distal end and a proximal end. A threaded insert is joined to an inner perimeter of the tack guide. The threaded insert includes an internally threaded section formed by helical members spaced axially from one another. The tack guide is more flexible towards the distal end and stiffer towards the proximal end. A portion of the guide between adjacent axially-separated cuts defines a link and each of the cuts serves as a joint between adjacent links.

### FIELD OF THE INVENTION

The present invention relates generally to tacker devices for applying surgical fasteners, such as rotary tacks, to tissues, such as for hernia repairs and the like, and particularly to a flexible tack guide.

### BACKGROUND OF THE INVENTION

A number of surgical, laparoscopic and endoscopic procedures require application of rotary tacks to tissues, such as for hernia repairs and the like.

Tackers for applying such rotary tacks are well known. In a typical tacker, a drive shaft is coupled to a trigger. Operating the trigger causes rotation of the drive shaft. Rotary tacks are rotatingly connected to the drive shaft and held in an articulated applicator arm. Operation of the trigger causes the drive shaft to rotate so as to distally advance the rotary tacks for deployment in tissue. Articulating cables are provided for articulating the applicator arm at different angles. The articulating cables are manipulated by a handle of the tacker.

The rotary tacks advance through the end of the applicator arm on account of the inner surface of the arm being internally threaded. The rotary tacks advance by a screwing action—the tacks behave like external threads that threadingly advance through the internal threads.

However, in many surgical procedures it would be desirable for the tacker applicator arm to be able to bend in the patient's body in order to reach certain places. The problem in the prior art is that the threaded advancement of tacks is impeded if the tacker applicator arm bends to a curved position.

### SUMMARY OF THE INVENTION

The present invention seeks to provide an improved tack guide for surgical tackers, as is described more in detail hereinbelow. In particular, the present invention seeks to provide a flexible tack guide that allows for threaded advancement of tacks even when the guide is bent to a curved position. The guide is made of a specially cut tube with a threaded insert welded or otherwise affixed in the inside of the tube.

There is thus provided in accordance with a non-limiting embodiment of the present invention a flexible tack guide including a tube with a wall thickness and having a distal end and a proximal end, and a threaded insert joined to an inner perimeter of the tack guide, the threaded insert including an internally threaded section formed by helical members spaced axially from one another. A portion of the guide between adjacent axially-separated cuts defines a link and each of the cuts serves as a joint between adjacent links. The tack guide may be more flexible towards the distal end and stiffer towards the proximal end.

In accordance with an embodiment of the present invention a pattern of axially-separated cuts are formed through the wall thickness of the tack guide. Each of the cuts may include two parallel, axial cuts separated by a non-cut portion. The non-cut portions may increase in size with increasing distance from the distal end of the tack guide. The proximal end of the tack guide may have a keyed shape.

In accordance with an embodiment of the present invention the threaded insert is not joined over its entire length to the inner perimeter of the tack guide.

### BRIEF DESCRIPTION OF THE DRAWINGS

The present invention will be understood and appreciated more fully from the following detailed description taken in conjunction with the drawings in which:

FIG. 1 is a simplified pictorial illustration of a flexible tack guide mounted at the end of an applicator shaft of a tacker, constructed and operative in accordance with an embodiment of the present invention;

FIG. 2 is a simplified pictorial illustration of the flexible tack guide;

FIG. 3 is a simplified sectional illustration of the flexible tack guide; and

FIG. 4 is a simplified illustration of crimping cuts in the tack guide, in accordance with an embodiment of the invention.

### DETAILED DESCRIPTION OF EMBODIMENTS

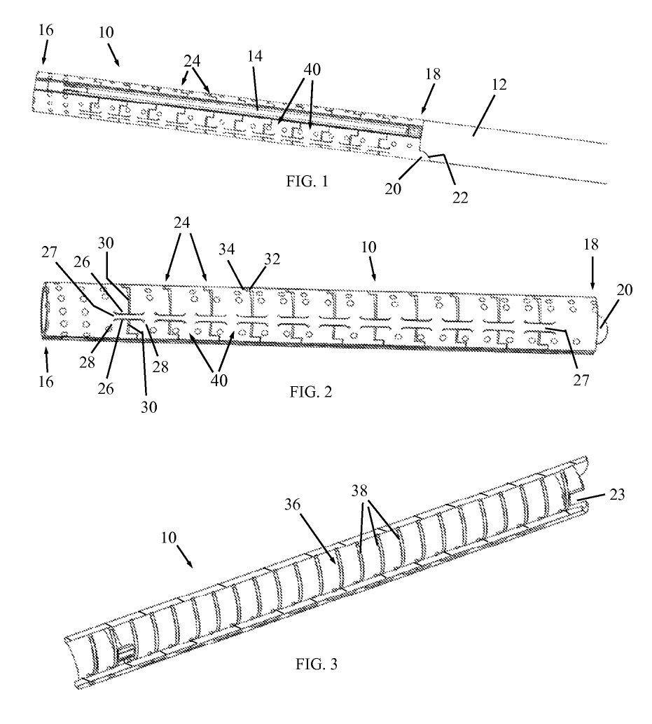

Reference is now made to FIG. 1, which illustrates a flexible tack guide 10, constructed and operative in accordance with a non-limiting embodiment of the present invention. The flexible tack guide 10 may be made of a medically safe material, such as but not limited to, stainless steel, titanium, nitinol and others.

The flexible tack guide 10 is a tube (also referred to as tube 10) with a wall thickness (of any suitable dimension) configured to mount on the distal end of an applicator shaft 12 of a tacker. The guide 10 may be welded at its proximal end 18 to the distal end of shaft 12 or may be affixed in any other suitable manner. One or more articulating cables 14 extend through the applicator shaft 12 and are connected to flexible tack guide 10, such as at a distal end 16 of guide 10. The articulating cables 14 are manipulated by a handle of the tacker (not shown) as is known in the art.

As seen in FIG. 1, the proximal end 18 of flexible tack guide 10 may have a keyed shape, such as a protruding member 20 which fits with a complementary-shaped recess 22 formed in the distal end of applicator shaft 12. Additionally or alternatively, the keyed shape may include a recess 23 (FIG. 3) formed in the flexible tack guide 10, which fits with a complementary-shaped protrusion (not shown) in the distal end of applicator shaft 12. The keyed shape ensures proper rotational registration with the applicator shaft 12, so that the articulating cables 14 pass properly through shaft 12 to guide 10.

As seen best in FIG. 2, a pattern of axially-separated cuts 24 are formed in or through the wall thickness of tack guide 10 (that is, the cuts 24 can be blind cuts or through cuts or a combination of both). Each cut 24 includes two parallel, axial (that is, longitudinal, which is horizontal in the drawing) cuts 26 separated by a non-cut portion 27, which serves as a beam. The ends of cuts 26 terminate in tilted cuts 28 which are tilted away from the non-cut portion 27. Each cut further comprises two partially circumferential cuts 30 that extend circumferentially outwards from a middle portion of cuts 26 away from the non-cut portion 27. Each of the ends of the circumferential cuts 30 may continue as a short axial cut 32 followed by a short circumferential cut 34. The pattern of cuts 24 may be made by laser cutting or any other suitable technique. In some embodiments, there may be no non-cut portion between some of the cuts 26.

The portion of the tube 10 between adjacent axially-separated cuts 24 defines a link 40. Each cut 24 serves as a joint or pivot between adjacent links 40. In other words, the guide 10 bends by means of links 40 bending at cuts (joints) 24. The series of non-cut portions 27 form a backbone or spine which is stiffer than the rest of the circumference of the links 40.

In one embodiment, all the links 40 have equal stiffness. In another embodiment, some or all of the links 40 do not have equal stiffness. The unequal stiffness may be achieved in several ways. For example, in one embodiment, the guide 10 is more flexible towards the distal end 16 of guide 10 and stiffer towards the proximal end 18. One way of accomplishing this is shown in FIG. 2. The non-cut portions 27 increase in size with increasing distance from the distal end 16 of guide 10. This makes guide 10 more flexible towards the distal end 16 and stiffer towards the proximal end 18. In another embodiment, some or all of the non-cut portions 27 have different axial lengths; this provides unequal stiffness which may be engineered for a particular application. In yet another embodiment, unequal stiffness may be achieved by cutting inside the non-cut portions 27 or other parts of the tube 10.

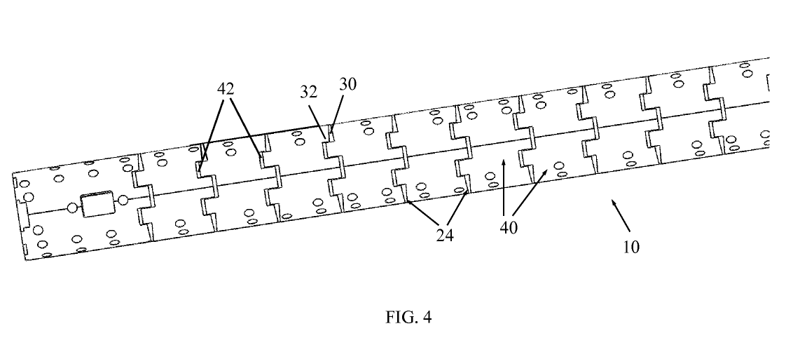

When the tube 10 bends back to a straight position from a bent position, it is possible, due to the cut-out material, for the tube to over-shoot the straight position and bend in the opposite direction. In order to ensure that the tube bends back to the straight position, some of the cuts, such as the axial cuts 32 and the circumferential cuts 30 and 34, may be crimped or partially filled with filling or welding material, as indicated in FIG. 4 by reference numeral 42. This limits the amount the links can bend back and thus reduces or eliminates the possibility of over-shooting the straight position.

As seen in FIG. 3, a threaded insert 36 is affixed inside the tack guide 10. The threaded insert 36 is an internally threaded section formed by helical members 38 spaced axially from one another. Rotary tacks (not shown) can threadedly advance through threaded insert 36. The threaded insert 36 may be made of stainless steel or any other suitable material. Threaded insert 36 may be welded, bonded or otherwise joined to the inner perimeter of tack guide 10, but is not joined over its entire length, rather only at a few points. (Possible welding points are shown as small circles in FIG. 2.) In this manner, the threaded insert 36 remains flexible so as to bend and yield to the bent shape of tack guide 10. The helical members 38 are sufficiently flexible to allow for the small changes in spacing between adjacent members 38 so that the rotary tacks advance smoothly even with the tube (guide 10) bent to a curved shape.

What is claimed is:

1. A tacker device comprising:
a flexible tack guide comprising a tube with a wall thickness and having a distal end and a proximal end; and
a threaded insert joined to an inner perimeter of said tack guide, said threaded insert comprising an internally threaded section formed by helical members spaced axially from one another, wherein a pattern of axially-separated cuts are formed in or through the wall thickness of said tack guide, and wherein a portion of said guide between adjacent axially-separated cuts defines a link and each of said cuts serves as a joint between adjacent links.

2. The tacker device according to claim 1, wherein said tack guide is more flexible towards the distal end and stiffer towards the proximal end.

3. The tacker device according to claim 2, wherein each of said cuts comprises two parallel, axial cuts separated by a non-cut portion.

4. The tacker device according to claim 3, wherein said non-cut portions increase in size with increasing distance from the distal end of said tack guide.

5. The tacker device according to claim 3, wherein ends of said parallel, axial cuts terminate in tilted cuts which are tilted away from said non-cut portion.

6. The tacker device according to claim 3, wherein each of said cuts further comprises two partially circumferential cuts that extend circumferentially outwards from a middle portion of said parallel, axial cuts away from said non-cut portion.

7. The tacker device according to claim 6, wherein each of ends of the circumferential cuts continues as an axial cut followed by a circumferential cut.

8. The tacker device according to claim 1, wherein one or more articulating cables are connected to said tack guide.

9. The tacker device according to claim 1, wherein the proximal end of said tack guide has a keyed shape.

10. The tacker device according to claim 1, wherein said threaded insert is not joined over its entire length to the inner perimeter of said tack guide.

11. The tacker device according to claim 1, wherein all the links have equal stiffness.

12. The tacker device according to claim 1, wherein some or all of the links do not have equal stiffness.

13. The tacker device according to claim 3, wherein some or all of the non-cut portions have different axial lengths.

14. The tacker device according to claim 1, wherein some of the cuts are crimped or partially filled with a filling material.
